(19) United States
(12) Patent Application Publication
(10) Pub. No.: US 2008/0274987 A1
(43) Pub. Date: Nov. 6, 2008

(54) Novel alpha glucosidase activator, pulicarside 1

(75) Inventors: Viqar Uddin Ahmad, Karachi (PK); Muhammad Iqal Choudhary, Karachi (PK); Nasir Khan, Sahiwal (PK); Shamsun Nihar Khan, Dhaka (PK)

Correspondence Address: SARFARAZ K. NIAZI, 20 RIVERSIDE DRIVE, DEERFIELD, IL, 60015, US

(73) Assignee: HEJ Research Institute, Karachi (PK)

(21) Appl. No.: 11/422,833

(22) Filed: Jun. 7, 2006

(51) Int. Cl. A61K 31/7028; C07H 15/00; A61P 3/04; A61P 35/00; A61P 31/10; A61P 37/02

(52) U.S. Cl. 514/27; 536/4.1

(57) ABSTRACT

A new ent-kaurane type diterpene glucoside, pulicarside (1) was isolated from Pulicaria undulate with a strong α-glucosidase promoter activity; a large number of clinical applications are provided.

### BACKGROUND

Glucosidase enzymes are involved in several biological processes such as the intestinal digestion, the biosynthesis of glycoproteins and the lysosomal catabolism of the glycoconjugates (Homonojirimycin isomers and N-alkylated homonojirimycins: structural and conformational basis of inhibition of glycosidases. Asano N, Nishida M, Kato A, Kizu H, Matsui K, Shimada Y, Itoh T, Baba M, Watson A A, Nash R J, Lilley P M, Watkin D J, Fleet G W., J Med. Chem. 1998 Jul. 2; 41(14):2565-71). Intestinal α-glucosidases are involved in the final step of the carbohydrate digestion to convert these into monosaccharides which are absorbed from the intestine. See Scheme 1

Scheme 1. Schematic diagram of enzymatic degradation of poly- and oligosaccharides and sucrose by Intestinal α-glucosidase.

As a result of the catalysis produced by α-glucosidase enzyme in the final step in the digestive process of carbohydrates, its inhibitors can retard the uptake of dietary carbohydrates and suppress postprandial hyperglycemia, and could be useful to treat diabetic and/or obese patients [Novel α-glucosidase Inhibitors with a tetrachlorophthalimide Skeleton., S. Sou, S. Mayumi, H. Takahashi, R. Yamasak, S. Kadoya, M. Sodeoka, and Y. Hashimoto, Bioorg. Med. Chem. Lett., 2000, 10, 1081]. The α-glucosidase inhibitors are effective in lowering the insulin release, insulin requirement and some can lower plasma lipids. The acarbose is a very widely prescribed drug in the management of the type II diabetes.

In addition, they have also been used as antiobesity drugs, fungistatic compounds, insect antifeedents, antivirals and immune modulators [Glycosidase inhibitors and their chemotherapeutic value, Part 1. el Ashry E S, Rashed N, Shobier A H., Pharmazie. 2000 April; 55(4):251-620]. The antiviral activity due to inhibition of α-glucosidase results form abnormal functionality of glycoproteins because of incomplete modification of glycans. Suppression of this process is the basis of antiviral activity [A glucosidase-Inhibitors as potential broad based antiviral agents, Anand Mehta, Nicole Zitzmann, Pauline M. Rudd, Timothy M. Block, Raymond A. Dwek, Febs Letters 430 (1998)17-22] and decrease in growth rate of tumors [Inhibition of experimental metastasis by an alpha-glucosidase inhibitor, 1,6-epi-cyclophellitol. Atsumi S, Nosaka C, Ochi Y, linuma H, Umezawa K. Cancer Res. 1993 Oct. 15; 53(20):4896-9]. Examples of antitumor agents which are enzyme activators and inducers include p-propenylanisole, LKT-B6801 (Brassinin), LKT-B6998 (Bryostatin 1) and LKT-B6999 (Bryostatin 2).

Whereas a large volume of research has been conducted on α-glucosidase inhibitors and several commercial products introduced over the past several decades, the research on the activators of this enzyme has been minimal despite the realization that the α-glucosidase promoters may be of interest in cases of deficiency of this enzyme in a large variety of conditions. These include the hypoglycemic condition, increasing the calorific value of foods, biosynthesis and normal embryogenesis in plants [U.S. Pat. No. 4,449,997 to Iwamura, et al. May 22, 1984 for a Plant growth regulator], enhanced viral growth, glycoprotein formation [An Evaluation of pathogenicity and treatment using Glycobiology, Mc Gill Journal of Medicine Vol. 7, No. 2, 2004], cellulose biosynthesis and tissue culture [α-glucosidase-1 is required for cellulose biosynthesis and morphogenesis in Arabidopsis, The journal of cell biology, Vol 156, No. 6, 2002], seed storage protein values [Arabidopsis glucosidase I mutant reveal a critical role of N-glycan trimming in seed development. The EMBO Journal Vol. 20 No. 5 pp 1010-1019, 2001], operational stability of the enzyme [Stabilization of immobilized carbohydrate-metabolizing enzymes. Maruo Shigeaki; Patent Classifications Main IPC: C12no11-00; Biosensors the stability problem, Tim D. Gibson Analusis, 1999, 27, No. 7], and in the treatment of genetic disorders like the Pompe's disease [Evasion of immune responses to introduced human acid alpha-glucosidase by liver-restricted expression in glycogen storage disease type II. Franco L M, Sun B, Yang X, Bird A, Zhang H, Schneider A, Brown T, Young S P, Clay T M, Amalfitano A, Chen Y T, Koeberl D D., Mol. Ther. 2005 November; 12(5):876-84]; it can also be used to enhance the effect of exogenous source of α-glucosidase [Myozyme (recombinant α-glucosidase approved by the US FDA in April 2006].

The activation of α-glucosidase enzyme is related to prolongation of its stability. The stabilization of enzymes and other proteins is of great interest in a number of application areas where shelf life and stability during operation are required. Stability categorized in two types: a. shelf stability and b. operational stability. The shelf stability involves activity retained in an enzyme or protein, diagnostic or device when stored under specified conditions after manufacturing. This parameter is vital for the commercialization of labile materials that degrade over time. The operational stability involves retention of the activity of a protein or enzyme when in use. This is often the most quoted parameter in the development of biosensors. The probable mechanism of the inactivation of the enzymes include unfolding (denaturation), loss of co-factor (FAD: Haem: PQQ), protein aggregation, poisoning irreversible inhibition, proteolysis, and substrates for microorganisms.

The enzymes can be stabilized in the following ways: physical entrapment, chemical modification-graft polymers, cross-linking bifunctional reagents, immobilization on supports-electrodes and protein engineering. Additives often used in stabilizing enzymes include salts and buffers, metal ions-magnesium, calcium, polymers-neutral/charged, solvents and water modifiers such as sugar and polyhydric alcohols. The polyalcohols include sugars, sugar alcohols and other more exotic substituted sugar molecules. These polyhydric molecules modify the water environment surrounding a protein competing for and replacing the free water thus modifying the hydration shell of the protein in question. This modified hydration shell confers protection the protein while maintaining 3D structure and biological activity with time. The storage of biological materials in the presence of these polyalcohol molecules allows storage in solution and in dehydrated state with vastly deceased levels of biological activity with time. [Biosensors the stability problem, Tim D. Gibson Analusis, 1999, 27, No. 7].

Examples of commercially available enzyme activators include: Adenylate Cyclase Toxin, Bordetella pertussis, Recombinant, E. coli, Cholera Toxin, A Subunit, Vibrio cholerae, Type Inaba 569B, Cholera Toxin, Vibrio cholerae, Type Inaba 569B, Cholera Toxin, Vibrio cholerae, Type Inaba 569B, Azide Free, L-(−)-Epinephrine-(+)-bitartrate, Forskolin, 1,9-Dideoxy-, Coleus forskohlii, Forskolin, 7-Deacetyl-, Forskolin, 7-Deacetyl-7-[O—(N-methylpiperazino)-α-butyryl]-, Dihydrochloride, Forskolin, 7-Deacetyl-7-O-hemisuccinyl-, Forskolin, Coleus forskohlii, Isoproterenol, Hydrochloride, PACAP 27 Amide, Ovine, PACAP 38, Ovine. Whereas it is relatively easy to look for α-glucosidase inhibitors, there is no certain method of determining if a compound will act as α-glucosidase activator, given the many mechanism by which the process of activation works as enumerated above. In the present invention reported, a surprising discovery was made when a novel α-glucosidase activator was found in the Pulicaria undulate (herb) that belongs to the family Asteraceae (Compositae). This compound has never before reported in the prior art.

Pulicaria undulata (herb) belongs to the family Asteraceae (Compositae), which is a largest family of flowering plants. Plants of this family are found in frigid, temperate subtropical and tropical zones of Africa and Asia. The genus Pulicaria has eleven species, distributed in tropical and temperate regions of Pakistan [Flora of West Pakistan, E. Nasir, 1972, no. 20, pp. 770]. The plants of this genus are used in traditional medicine as tonic and substituted for tea, antispasmodic, hypoglycemic and as ingredients of perfume [D-Carvotanacetone from Pulicaria Undulata, Kamal El Din A, Yousif G, Ishag K E, El Egami A A, Mahmoud E N, Abu Al Futuh I M. Fitoterapia. 1992; 63:281] Aerial parts of Pulicaria undulata are used for antibacterial purpose [Antibacterial Properties of Essential Oils from Nigella Sativa Seeds (Cymbopogon Citratus) Leaves and Pulicaria Undulata Aerial Parts., Kamali H H, Ahmed A H, Mohammed A S, Yahia A A M, El Tayeb I, Ali A A. Fitoterapia, 1998; 69:77-78]. Literature survey showed some reports on essential oils [Isolation and antimicrobial activity of two phenolic compounds from Pulicaria odora L. Ezoubeiri A, Gadhi C A, Fdil N, Benharref A, Jana M, Vanhaelen M., J. Ethnopharmacol. 2005 Jun. 3; 99(2):287-92.], terpenoids and flavonoids [Isolation of dihydroflavonol from Pulicaria undulate (L.) Kostel. Khafagy S M, Metwally A M, Omar A A., Pharmazie. 1976; 31 (9):649]

The present invention deals with the isolation and characterization of a new ent-kaurane type diterpene glucoside, pulicarside (1), along with three known terpenoids, paniculoside IV (2), ent-16,17-dihydroxy-(−)-kauran-19-oic acid (3) [X. Jiang, M. Yunbao, X. Yunlong, Phytochemistry 1992, 31, 917], and 2α-hydroxy alantolactone (4) [F. Bohlmann, P. K. Mahanta, J. Jakupovic, R. C. Rastogi, A. A. Natu, Phytochemistry 1978, 17, 1165] (FIG. 1).

FIG. 1. Structure of compound 1 [pulicarside (1)]

### EXPERIMENTAL

General Analytical Instrumentation: TLC. Kieselgel F₂₅₄ (0.25 mm: Merck). Column chromatography (CC): silica gel (70-230 mesh, Merck), flash chromatography (FC): silica gel (230-400 mesh, Merck). Optical rotation. Jasco DIP-360 digital polarimeter. UV Spectra: Hitachi-UV-3200 spectrophotometer. IR spectra: Jasco-320-A spectrophotometer. ¹H-NMR, ¹³C-NMR, COSY, HMQC and HMBC Spectra. Bruker spectrometer. EI-MS and FAB-MS spectra: JMS-HX-110 spectrometer.

Plant material. The plant Pulicaria undulata L. (Asteraceae) was collected from Loralai, Balochistan (Pakistan), and identified by Prof. Rasool Bakhsh Tareen, Department of Botany, Balochistan University, Quetta, Pakistan. A voucher specimen (no. 1437a) has been deposited at the herbarium of the Department of Botany, Balochistan University, Quetta, Pakistan.

Extraction and purification. The shade-dried ground plant material (whole plant) (30 kg) was exhaustively extracted with methanol at room temperature. The extract was evaporated to yield the residue (753 g). The whole residue was dissolved in water and partitioned with hexane, chloroform, ethyl acetate and n-butanol. The ethyl acetate soluble extract (182 g) was subjected to column chromatography (silica gel, Hexane/CHCl₃ mixtures of increasing polarity, CHCl₃, CHCl₃/MeOH mixtures of increasing polarity) and fifteen fractions (1-15) were collected. Compound 2 (17.4 mg) was obtained from Fr. 9 when it was subjected to FC (silica gel, CHCl₃/MeOH (15:85). Compound 1 (22.1 mg) was obtained from Fr. 8 when it was subjected to FC (silica gel, CHCl₃/MeOH (10:90)). The chloroform soluble fraction (232 g) was submitted to columb chromatography (silica gel, Hexane/CHCl₃ mixtures of increasing polarity) and twenty fractions (1-20 were collected. Compound 4 (32.6 mg) was obtained from Fr. 7 when it was flash chromatographed (FC) over silica gel (EtOAc/hexane (12:88)). Similar to 4, compound 3 (12.4 mg) was obtained from Fr. 12 (EtOAc/hexane (45:55).

Acidic hydrolysis of 1. Compound 1 (5 mg) was refluxed with 0.5 N HCl (10 ml) for 2 h. After neutralization with NH₄OH, it was extracted with n-butanol. The n-butanol fraction was evaporated under reduced pressure to give glucoside without acetonyl moiety, ¹H-NMR data of which were identical with paniculoside IV (16, β-17-hydroxy-ent-1-α-auzan 19-O-D-glucopyranosyl ester [K. Yamasaki, H. Kohada, T. Kobayashi, N. Kaneda, R. Kasai, O. Tanaka, K. Nishi, Chem. Pharm. Bull. 1977, 25, 2895].

Basic hydrolysis of 1. Compound 1 (5 mg) was refluxed with 5% aqueous KOH solution (10 ml) for 2 h. The mixture was then neutralized with a dilute HCl solution and extracted with n-butanol (3×6 ml). The combined n-butanol fractions were evaporated to gave aglycone having similar ¹H-NMR data as that of already reported ent-16,17-acetonyl-(−)-kauran-19-oic acid [M. S. Correa, G. M. S. P. Guilhon, L. M. Conserva, Fitoterapia 1998, LXIX, 277]. The aqueous layer was also evaporated under reduced pressure and analyzed for sugar by thin layer chromatography and compared with authentic sample.

Pulicarside (=β-D-glucopyranosyl ent-16,17-acetonyl-(−)-kauran-19-oate; 1) is a white powder with following properties: [α]_{D}²⁵=−158.9 (c=0.15). IR: 2958, 2883, 2862, 1727, 1630, 1451, 1372, 1300, 1261, 1214, 1162, 1073, 1024. ¹H- and ¹³C-NMR: see Table 1. FAB-MS (pos.): 539 ([M+H]+). HR-FAB-MS (pos.): 539.3214 ([M+H]⁺, C₂₉H₄₇O₉; calc. 539.3220). See Table 1. The absorption band in the IR spectrum at 1727 cm⁻¹ showed a carbonyl function in the molecule. The positive FAB mass spectrum showed a quasimolecular ion peak at m/z 539 [M+H]⁺ and its molecular formula (C₂₉H₄₆O₉) was deduced from ¹³C-NMR and mass spectral data. The ¹³C-NMR spectrum showed 29 carbons which were resolved through DEPT experiment into four methyls, eleven methylenes, eight methines and six quaternary carbons. Out of 29 carbons, 20 were attributed to the diterpene skeleton, 6 to a hexose and three to an acetonoyl moiety. Five characteristic oxygenated methines and a methylene at δ 95.6 (5.40, H-anomeric), 74.1, 78.72, 71.1, 78.74, and 62.4, respectively, indicated a hexose unit in the molecule. The correlation in the HMBC spectrum between an anomeric proton and a carbonyl carbon (δ·178.3) showed that glycone was connected to aglycone through ester function. The signals for hexose were consistent with β-D-glucose [K. Yamasaki, H. Kohada, T. Kobayashi, N. Kaneda, R. Kasai, O. Tanaka, K. Nishi, Chem. Pharm. Bull. 1977, 25, 2895]. Four singlets, each of 3-protons integration were present at δ· 0.96, 1.21, 1.30, and 1.34 in the ¹H-NMR spectrum. Their associated carbon signals in the ¹³C-NMR spectrum at δ· 16.6, 29.0, 27.0, and 27.2, respectively revealed the presence of four tert-methyls, out of which two were related to diterpene skeleton and other two were of acetonyl moiety. After assigning the ¹H- and ¹³C-NMR chemical shifts (Table 1) with the help of HMQC, HMBC and COSY spectra (FIG. 1), the structure of pulicarside (1) was elucidated as β-D-glucopyranosyl-ent-16,17-acetonyl-(−)-kauran-19-oate. Since acetone was not used throughout the extraction and isolation procedure, which ruled out the possibility of an artifact. As a result of acidic hydrolysis of 1, a glucoside without acetonyl moiety was obtained, having identical NMR data with paniculoside IV [K. Yamasaki, H. Kohada, T. Kobayashi, N. Kaneda, R. Kasai, O. Tanaka, K. Nishi, Chem. Pharm. Bull. 1977, 25, 2895.]. The basic hydrolysis of 1 produced an aglycone whose data were found to similar with already reported ent-16,17-acetonyl-(−)-kauran-19-oic acid (3) [K. Yamasaki, H. Kohada, T. Kobayashi, N. Kaneda, R. Kasai, O. Tanaka, K. Nishi, Chem. Pharm. Bull. 1977, 25, 2895.

TABLE 1
¹H— and ¹³C— NMR Data of Pulicarside (1).
At 300 and 75 MHz; δ in ppm, J in Hz.

| Atom | δ(H) | δ(C) |
|---|---|---|
| H—C(1) | 0.83 | 41.9 |
| | 1.82 | |
| H—C(2) | 1.47 | 20.1 |
| | 1.93 | |
| H—C(3) | 1.47 | 39.1 |
| | 2.03 | |
| C(4) | | 45.1 |
| H—C(5) | 1.07 | 58.6 |
| H—C(6) | 1.89 | 23.1 |
| | 2.03 | |
| H—C(7) | 2.02 | 39.3 |
| C(8) | | 45.7 |
| H—C(9) | 1.01 | 56.8 |
| C(10) | | 40.8 |
| H—C(11) | 1.38 | 20 |
| | 1.62 | |
| H—C(12) | 1.38 | 27.9 |
| | 1.59 | |
| H—C(13) | 2.06 | 47.1 |
| H—C(14) | 1.43 | 43 |
| | 1.55 | |
| H—C(15) | 1.69 (d, J = 15.2) | 57.6 |
| | 1.78 (d, J = 15.2) | |
| C(16) | | 90.6 |
| H—(17) | 3.94 (d, J = 8.7) | 70.9 |
| | 4.07 (d, J = 8.7) | |
| H—C(18) | 1.21 s | 29 |
| C(19) | | 178.3 |
| H—C(20) | 0.96 s | 16.6 |
| H—C(1)′ | 1.31 s | 27 |
| C(2)′ | | 109.6 |
| H—C(3)′ | 1.34 s | 27.2 |
| H—C(1)″ | 5.40 (d, J = 7.7) | 95.6 |
| H—C(2)″ | 3.29-3.39 | 74.1 |
| H—C(3)″ | 3.29-3.39 | 78.72 |
| H—C(4)″ | 3.29-3.39 | 71.1 |
| H—C(5)″ | 3.29-3.39 | 78.74 |
| H—C(6)″ | 3.69 (dd, J = 12.1, 3.1) | 62.2 |
| | 3.69 (br. d, J = 12.1) | |

At 300/75 MHz; δ in ppm, J in Hz; Assigned on the basis of HMQC; In (D4) Methanol.

FIG. 2. Key 2D NMR correlations for compound 1 [pulicarside (1)]

### Activity Testing

α-Glucosidase (E.C.3.2.1.20) enzyme inhibition assay was performed according to the slightly modified method of Matsui et al. α-glucosidase (E.C.3.2.1.20) from Saccharomyces species, purchased from Wako Pure Chemical Industries Ltd. (Wako 076-02841). The enzyme inhibition was measured spectrophotometrically at pH 6.9 and at 37° C. using 0.7 mM p nitrophenyl-α-D-glucopyranoside (PNP-G) as a substrate and 500 m units/mL enzyme, in 50 mM sodium phosphate buffer containing 100 mM NaCl. 1-Deoxynojirimycin (0.425 mM) and acarbose (0.78 mM) were used as positive control. The increment in absorption at 400 nm, due to the hydrolysis of PNP-G by α-glucosidase, was monitored continuously on microplate spectrophotometer (Spectra Max Molecular Devices, USA).

[T. Matsui, C. Yoshimoto, K. Osajima, T. Oki, and Y. Osajima. Biosci. Biotech. Biochem., 1996, 60, 2019].

TABLE 1
Result of In vitro quantitative studies on
compounds 1-3 against α-glucosidase.

| Name of Substance | IC₅₀ ± SEM [μM] |
|---|---|
| Pulicarside (1) | Enhancer |
| Paniculoside IV (2) | 406.7 ± 20 |
| Eent-16,17-dihydroxy-(−)-kauran-19- | 62.2 ± 0.00 |
| oic acid (3) | |
| 1-Deoxynojirimycin^{(a)} | 425 ± 8.14 |
| Acarbose^{(a)} | 780 · 0.028 |

^{(a)}positive control for α-glucosidase.

Several unique observations were made during the studies conducted on Compound 1 activity. For example, it was observed that the enzyme generally looses its activity during the operational period. We have a set of experiment to see how the enzyme lost its activity with almost all activity gone within one hour. (Table 2).

TABLE 2
Rate of enzyme reaction as a function of time (μM
of alpha-glucose from the p-nitrophenyl at 400 nm) and
concentration of Compound 1 as a function of time.

| | Concentration of Compound 1 | | | | |
|---|---|---|---|---|---|
| Incubation | | 0.05 | | | |
| (min) | 0 mM | mM | 0.10 mM | 0.20 mM | 0.40 mM |
| 4 | 11.18 | 18.61 | 19.94 | 20.89 | 21.15 |
| 24 | 3.69 | 8.7 | 10.38 | 18.27 | 19.68 |
| 45 | 0.087 | 3.85 | 4.64 | 13.11 | 14.8 |
| 66 | 0.091 | 1.45 | 1.62 | 10.13 | 15.01 |

Pulicarside (1) showed a very interesting behavior towards the α-glucosidase enzyme. At all the concentrations and time intervals it enhanced the enzymatic activity, as compared to the negative control. As a result we conclude that the activity of Compound 1 is concentration dependent. The mechanism postulated here includes enhancement of stability of the enzyme to react with the substrate, which is not observed in negative control in the absence of this compound 1. From the structures it is clearly evidenced that the methylene-dioxy ring and as well as the sugar molecule is responsible for the enzyme activation. When the ring is cleaved in case of the compound, 2 (pulicarside IV) showed inhibitory effect on the enzyme (IC₅₀ 406.7±20) and when the sugar molecule is replaced by the COO— group from the molecule in case of the compound, 3 (Ent-16,17-dihydroxy-(−)-kauran-19-oic acid) (IC₅₀62.2±0.008) the compound showed a promising inhibitory activity against the enzymes. So the COO— group is playing a crucial role for the inhibitory effect on the enzyme and the gugar molecule and methylene-dioxy ring is responsible for the activation of the enzyme.

### DESCRIPTION OF DRAWINGS

Scheme 1. Schematic diagram of enzymatic degradation of poly- and oligosaccharides and sucrose by intestinal alpha-glucosidase.

FIG. 1 Structure of compound 1 [pulicarside (1)]

FIG. 2. Key 2D NMR correlations for compound 1 [pulicarside (1)]

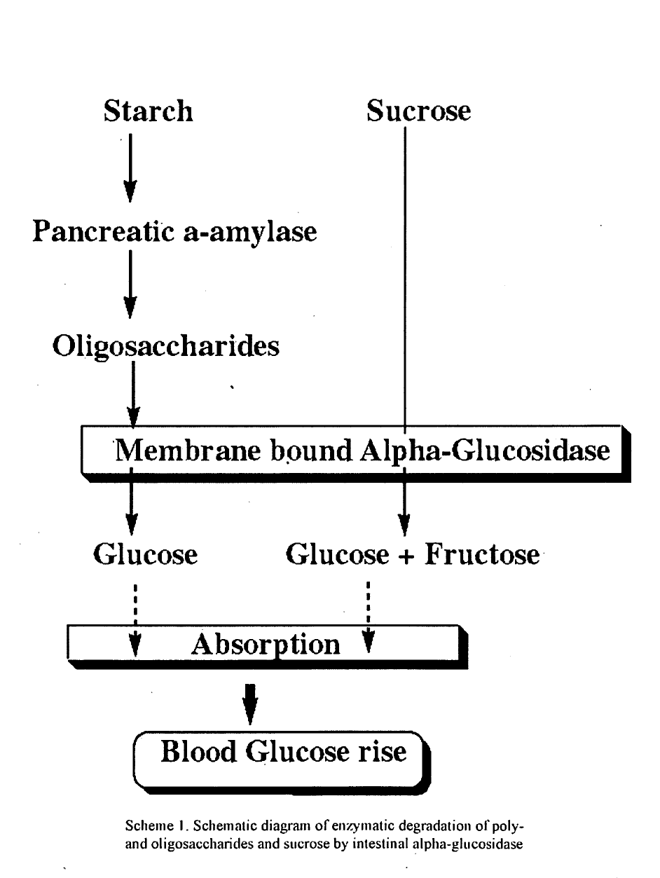

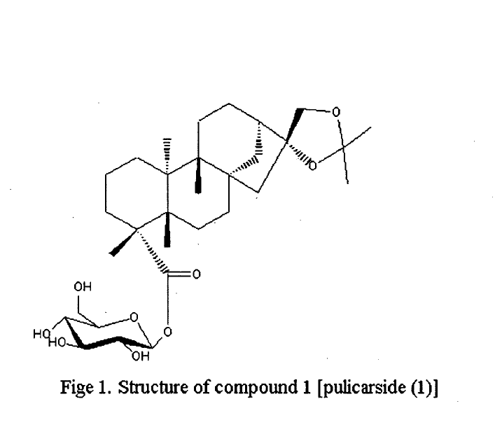

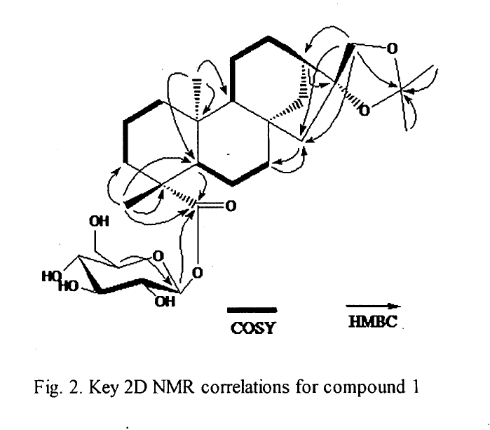

What is claimed is:

1. A compound having the formula (I), β-D-glucopyranosyl ent-16,17-acetonyl-(−)-kauran-19-oate; 1, and its pharmaceutically acceptable derivatives.

2. A pharmaceutical composition which comprises the compound of claim 1 and a pharmaceutically acceptable vehicle for administration to humans, animals and plants.
